(19) United States
(12) Patent Application Publication
(10) Pub. No.: US 2024/0299196 A1
(43) Pub. Date: Sep. 12, 2024

(54) PROSTHETIC LIMBS, PROSTHETIC LINERS, AND METHODS FOR MANAGING, CONFIGURING, AND USING PROSTHETIC LIMBS AND PROSTHETIC LINERS

(71) Applicants: Tan Tock Seng Hospital Pte Ltd, Singapore (SG); National Skin Centre (Singapore) Pte Ltd, Singapore (SG); Singapore University of Technology and Design, Singapore (SG)

(72) Inventors: Brian Trevor BINEDELL, Singapore (SG); Jia Yee TEO, Singapore (SG); Hong Yee LOW, Singapore (SG); Hong Liang TEY, Singapore (SG)

(73) Assignees: Tan Tock Seng Hospital Pte Ltd, Singapore (SG); National Skin Centre (Singapore) Pte Ltd, Singapore (SG); Singapore University of Technology and Design, Singapore (SG)

(21) Appl. No.: 18/277,071

(22) PCT Filed: Feb. 14, 2022
(86) PCT No.: PCT/SG2022/050070

§ 371 Date: Aug. 13, 2023

(30) Foreign Application Priority Data
Feb. 15, 2021 (SG) 10202101506T

(51) Int. Cl. A61F 2/78; A01G 25/00; A01G 31/02; A61F 2/50

(52) U.S. Cl. CPC A61F 2/7843; A01G 25/00; A01G 31/02; A61F 2002/501

(57) ABSTRACT

Embodiments relate to prosthetic limbs and liners. An embodiment of the prosthetic liner is housed in a prosthetic limb and includes a liner body. The liner body includes a first end for receiving a body part of a user. The first end includes a liquid-retentive portion. The liner body includes an inner surface. The inner surface contacts with the body part. The inner surface includes a plurality of microchannels. The microchannels are formed along the inner surface. The microchannels are connected at one end to the liquid-retentive portion. At least one of the plurality of microchannels is configured to receive liquid droplets and direct the received liquid droplets to the liquid-retentive portion. The liner body also includes an outer surface opposite to the inner surface. The outer surface faces an interior surface of the prosthetic limb and is provided in an interior cavity of the prosthetic limb.

### TECHNICAL FIELD

The present disclosure relates generally to prosthetic limbs and prosthetic liners. More particularly, the present disclosure relates to prosthetic limbs and prosthetic liners configured to direct, collect, and/or otherwise control or manage liquid introduced from, by, or as a result of a body part of a user (e.g., sweat) and/or external factors.

### BACKGROUND

Prosthetic liners are important for wearers of prosthetic limbs to help prevent negative effects from wearing the prosthetics for several hours at a time. Such negative effects may include friction against the hard prosthetic material and moisture buildup from sweat which is not only uncomfortable but may lead to problems such as abrasions, pain, and other skin damage.

Studies have found that more than 53% of prosthetic users feel discomfort due to excessive heat or sweating, and an increment of 1-2° C. is sufficient to trigger this discomfort. Conventional prosthetic liners focus on improving fit and comfort by using cushioning materials. However, the human thermoregulatory system may increase the rate of perspiration due to these liners creating a barrier to thermal transfer mechanisms.

### SUMMARY

The present disclosure relates generally to prosthetic liners, prosthetics, and/or the like, for addressing conventional problems, including those described above and in the present disclosure, and more specifically, example embodiments relate to improved prosthetic liners, prostheses, and/or the like, including those that improve or reduce discomfort and/or other negative effects normally encountered when using prostheses. For example, embodiments described herein relate to prosthetic liners for use with prostheses.

In an exemplary embodiment, a prosthetic limb assembly is described. The prosthetic limb assembly includes a prosthetic limb. The prosthetic limb includes an interior surface forming an interior cavity. The prosthetic limb assembly also includes a prosthetic liner. The prosthetic liner is configured to be housed in the interior cavity of the prosthetic limb. The prosthetic liner includes a liner body. The liner body is formed in a shape resembling a shape of at least a portion of the interior surface of the prosthetic limb. The liner body includes a first end for receiving an insertion of a body part of a user. At least a portion of the first end of the liner body includes a liquid-retentive portion. The liner body also includes a second end opposite to the first end. The liner body also includes an inner surface formed between the first and second ends. The inner surface of the liner body is configured to contact with at least a portion of the body part of the user. The inner surface of the liner body includes a plurality of microchannels. The plurality of microchannels is formed along the inner surface of the liner body. The plurality of microchannels is connected at one end to the liquid-retentive portion of the first end of the liner body. At least one of the plurality of microchannels is configured to receive liquid droplets and direct the received liquid droplets to the liquid-retentive portion of the first end of the liner body. The liner body also includes an outer surface opposite to the inner surface of the liner body. The outer surface of the liner body faces the interior surface of the prosthetic limb and provided in the interior cavity of the prosthetic limb.

In another exemplary embodiment, a prosthetic liner is described. The prosthetic liner is for use with a prosthetic limb. The prosthetic limb includes an interior surface forming an interior cavity. The prosthetic liner includes a liner body. The liner body is formed in a shape resembling a shape of at least a portion of the interior surface of the prosthetic limb. The liner body is configured to be housed in the interior cavity of the prosthetic limb. The liner body includes a first end for receiving an insertion of a body part of a user. At least a portion of the first end of the liner body includes a liquid-retentive portion. The liner body also includes a second end opposite to the first end. The liner body also includes an inner surface formed between the first and second ends. The inner surface of the liner body is configured to contact with at least a portion of the body part of the user. The inner surface of the liner body includes a plurality of microchannels. The plurality of microchannels is formed along the inner surface of the liner body. The plurality of microchannels are connected at one end to the liquid-retentive portion of the first end of the liner body. At least one of the plurality of microchannels is configured to receive liquid droplets and direct the received liquid droplets to the liquid-retentive portion of the first end of the liner body. The liner body also includes an outer surface opposite to the inner surface of the liner body. The outer surface of the liner body faces the interior surface of the prosthetic limb and is provided in the interior cavity of the prosthetic limb.

In another exemplary embodiment, a prosthetic liner is described. The prosthetic liner is for use with a prosthetic limb. The prosthetic limb includes a liner body. The liner body is formed in a shape resembling a shape of at least a portion of the interior surface of the prosthetic limb. The liner body is configured to be housed in the interior cavity of the prosthetic limb. The liner body includes a first end for receiving an insertion of a body part of a user. The liner body also includes a second end opposite to the first end. The liner body also includes an inner surface formed between the first and second ends. The inner surface of the liner body is configured to contact with at least a portion of the body part of the user. The inner surface of the liner body includes a plurality of microchannels. The plurality of microchannels are formed along the inner surface of the liner body. At least one of the plurality of microchannels is configured to receive and collect liquid droplets. The liner body also includes an outer surface opposite to the inner surface of the liner body. The outer surface of the liner body faces the interior surface of the prosthetic limb and is provided in the interior cavity of the prosthetic limb. The outer surface of the liner body includes a plurality of second microchannels. The plurality of second microchannels are formed along the outer surface of the liner body. At least one of the plurality of second microchannels is configured to receive and collect liquid droplets.

### BRIEF DESCRIPTION OF FIGURES

For a more complete understanding of the present disclosure, example embodiments, and their advantages, reference is now made to the following description taken in conjunction with the accompanying figures, in which like reference numbers indicate like features, and:

FIG. 1 is a top view of an example embodiment of a prosthetic liner for use with a prosthetic limb;

FIG. 2 is a front view of an example embodiment of a prosthetic liner for use with a prosthetic limb; and

FIG. 3 is a schematic view of an irrigation system in accordance with an alternative embodiment.

Although similar reference numbers may be used to refer to similar elements in the figures for convenience, it can be appreciated that each of the various example embodiments may be considered to be distinct variations.

Example embodiments will now be described with reference to the accompanying figures, which form a part of the present disclosure, and which illustrate example embodiments which may be practiced. As used in the present disclosure and the appended claims, the terms “embodiment,” “example embodiment,” “exemplary embodiment,” and “present embodiment” do not necessarily refer to a single embodiment, although they may, and various example embodiments may be readily combined and/or interchanged without departing from the scope or spirit of example embodiments. Furthermore, the terminology as used in the present disclosure and the appended claims is for the purpose of describing example embodiments only and is not intended to be limitations. In this respect, as used in the present disclosure and the appended claims, the term “in” may include “in” and “on,” and the terms “a,” “an,” and “the” may include singular and plural references. Furthermore, as used in the present disclosure and the appended claims, the term “by” may also mean “from,” depending on the context. Furthermore, as used in the present disclosure and the appended claims, the term “if” may also mean “when” or “upon,” depending on the context. Furthermore, as used in the present disclosure and appended claims, the words “and/or” may refer to and encompass any or all possible combinations of one or more of the associated listed items.

### DETAILED DESCRIPTION

In the following detailed description, reference is made to the accompanying drawings which form a part hereof. The processes and system described in the detailed description and drawings are for illustrative purposes and are not meant to be limiting. Other embodiments can be utilised and other changes can be made, without departing from the scope of the disclosure presented herein. In the present disclosure, the depiction of a given element or consideration or use of a particular element number in a particular figure or a reference thereto in corresponding descriptive material can encompass the same, an equivalent, or an analogous element or element number identified in another figure or description material associated therewith.

Prosthetic limbs (or prostheses), or the like, play an important part in helping those who have lost a limb to replace at least some functionality and/or appearance of such lost limbs. While prostheses indeed assist in replacing the function and/or appearance of lost limbs, users of prostheses oftentimes encounter varying degrees of discomfort and/or other negative effects resulting from the use of prostheses. For example, studies have found that more than 53% of prosthetic users encounter discomfort and/or other negative effects due as a result of heat or sweating, and an increment of merely 1-2° C. is sufficient to trigger this discomfort.

Present example embodiments relate generally to and/or include prosthetic liners, prostheses, and/or the like, for addressing conventional problems, including those described above and in the present disclosure, and more specifically, example embodiments relate to improved prosthetic liners, prostheses, and/or the like, including those that improve or reduce discomfort and/or other negative effects normally encountered when using prostheses. For example, embodiments described herein relate to prosthetic liners for use with prostheses.

It is to be understood that, while example embodiments are mostly described in the present disclosure as pertaining to prosthetic liners, the principles described in the present disclosure may also be applied beyond the context of prosthetic liners for use with prosthetics, such as liners for use with other wearable objects, gear, devices, or the like, and prostheses having liners formed as a unitary article, without departing from the teachings of the present disclosure.

Example embodiments will now be described below with reference to the accompanying figures, which form a part of the present disclosure.

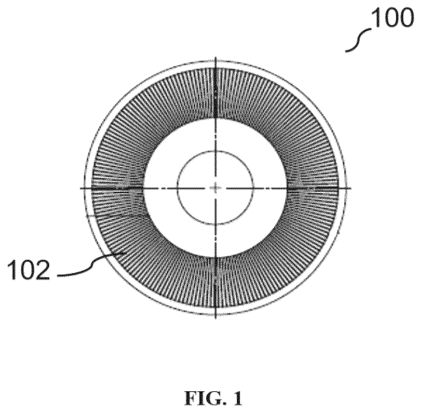

FIG. 1 illustrates an example embodiment of a prosthetic liner (e.g., prosthetic liner 100). The prosthetic liner 100 may be configurable or configured for use with a prosthetic limb (or prostheses, not shown) to address one or more conventional problems described above and in the present disclosure. For example, the prosthetic liner 100 may be provided in, provided on, and/or attached or secured to at least a part of a prosthetic in such a way that, when in use, the prosthetic liner 100 is adjacent to and/or contacts with a body part of a user. As a more specific but non-limiting example, the prosthetic liner 100 may be provided in, provided on, and/or attached or secured to an inner surface of a prosthetic arm, prosthetic leg, or the like.

An example embodiment of the prosthetic liner 100 may be formed in any one or more shapes and using any one or more types of materials that provide for an improvement to the fit and comfort of wearing the prosthetics. It is recognized in the present disclosure, however, that the human thermoregulatory system may increase the rate of perspiration when using prosthetics and/or prosthetic liners, which may result in creating a barrier, layer, or the like, to thermal transfer mechanisms and causing discomfort and/or other negative effects. Furthermore, friction against the prosthetic material, constant contact against one or more surfaces of the prosthetics, and/or continued sweat and/or moisture buildup may cause further discomfort and/or other negative effects, including abrasions, pain, skin rashes, and/or other skin damage. While various solutions and approaches may be used to address such discomfort and/or other negative effects users face when using prosthetics, such as the use of powders, creams, or the like, to protect the skin of prosthetic users, it is recognized in the present disclosure that such approaches remain insufficient, especially when addressing the problem of sweat and/or moisture buildup resulting from extended use of prosthetics (e.g., wearing of prosthetics for several hours at a time).

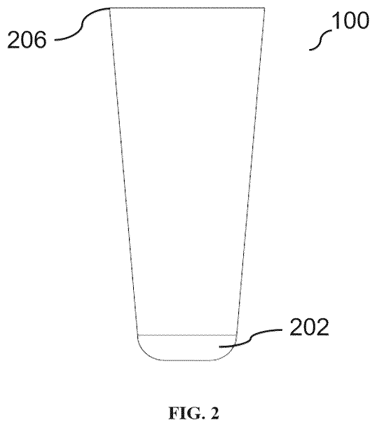

In an example embodiment, the prosthetic liner 100 can be formed in the shape resembling an interior of the prosthetic and a size suitable for the prosthetic liner 100 to be provided in, provided on, and/or attached or secured to the prosthetic and for the prosthetic liner 100 to be adjacent to and/or in contact with a body part of the user. As illustrated in FIG. 2, the prosthetic liner 100 includes a top section (e.g., top section 206), which may be formed as or having an open end (for insertion of a body part of the user). The prosthetic liner 100 may also include a bottom section (e.g., bottom section 202), which may be formed as a closed end. In some embodiments, the bottom section 202 may also be formed as an open or partially open end.

The prosthetic liner 100 also includes one or more side walls, or the like, formed between and secured to the top section 206 and bottom section 202 of the prosthetic liner 100. For example, a single continuous side wall may be formed between the top section 206 and bottom section 202, such as in example embodiments in which the prosthetic liner is formed in the shape of a cylinder, or the like. Each of the one or more side walls of the prosthetic liner 100 includes an inner surface directly facing, adjacent to, and/or in contact with a body part of the user. Furthermore, each inner surface of the prosthetic liner 100 includes at least a portion having a plurality of microchannels (e.g., microchannels 102), or the like. Furthermore, an inner surface of the bottom section 202 (e.g., when the bottom section 202 is formed as a closed end) may also include at least a portion having a plurality of the microchannels 102. As will be further described in the present disclosure, example embodiments of the microchannels 102 are configured to collect, direct, and/or otherwise control or manage liquid (e.g., sweat, moisture, water, etc.) introduced from, by, or as a result of the body part of the user and/or external factors (e.g., environment, humidity, etc.). FIG. 1 illustrates an example embodiment in which the entire inner surface of the side walls of the prosthetic liner 100 includes microchannels 102. In such a case, the prosthetic liner 100 may (or may not) be formed as a unitary article (e.g., a unitary cylindrical or other shaped liner having microchannels 102 formed throughout the entire inner surface, etc.). It is to be understood in the present disclosure, however, that in some embodiments, the prosthetic liner 100 may be formed in such a way that one or more portions of the inner surface of the prosthetic liner 100 may not include microchannels 102. For example, the prosthetic liner 100 may be formed via a plurality of quadrilateral shaped pieces (and/or other geometric shaped pieces), with at least one piece having microchannels 102 (e.g., for parts of the body part of the user that contacts with or is expected to contact with the prosthetic liner 100, etc.) and at least one piece not having microchannels 102 (e.g., for parts of the body part of the user that do not contact with or is not expected to contact with the prosthetic liner 100, etc.).

Each of the one or more side walls of the prosthetic liner 100 also includes an outer surface facing the opposite direction as the inner surface of the prosthetic liner 100. In some embodiments, the inner surface and outer surface are formed as separate walls, or the like. Each outer surface (or outer wall) of the prosthetic liner 100 may include at least a portion having a plurality of microchannels (e.g., microchannels 102), or the like. Furthermore, an outer surface of the bottom section 202 (e.g., when the bottom section 202 is formed as a closed end) may also include a portion having a plurality of the microchannels 102. As will be further described in the present disclosure, example embodiments of the microchannels 102 are configured to collect, direct, and/or otherwise control liquid (e.g., sweat, moisture, water, etc.) between the prosthetic liner 100 and the prosthetic limb coming from outside of the prosthetic limb. In example embodiments, the entire outer surface of the side walls of the prosthetic liner 100 may include microchannels 102. In such a case, the prosthetic liner 100 may (or may not) be formed as a unitary article (e.g., a unitary cylindrical or other shaped liner having microchannels 102 formed throughout the entire outer surface, etc.). It is to be understood in the present disclosure, however, that in some embodiments, the prosthetic liner 100 may be formed in such a way that one or more portions of the outer surface of the prosthetic liner 100 may not include microchannels 102. For example, the prosthetic liner 100 may be formed via a plurality of quadrilateral shaped pieces (and/or other geometric shaped pieces), with at least one piece having microchannels 102 formed on the outer surface and at least one piece not having microchannels 102 formed on the outer surface.

In an example embodiment, the microchannels 102 of the inner surface and/or outer surface of the prosthetic liner 100 may be formed using any one or more types/forms/compositions of soft hydrophilic material, including those having polyurethane-like properties. In an example embodiment, some, or all of the microchannels 102 of the inner surface and/or outer surface of the prosthetic liner 100 may be formed from materials having a shore hardness of about 40 A. In an example embodiment, a dimension of each microchannel 102 may be not more than about 0.2 mm (e.g., a diameter or width of not more than about 0.2 mm).

In use, the prosthetic liner 100 may be rolled on from the bottom section 202 to the top section 206. The inner surface of the prosthetic liner 100 having microchannels 102 formed on at least a portion of the inner surface will be in contact with the skin of the user. The prosthetic liner 100 may be removed by unrolling the prosthetic liner 100 from the top section 206 to the bottom section 202 and can be repeatedly washed like regular laundry.

During use, a wearer of the prosthetic liner 100 will secrete sweat throughout the day. The microchannels 102 of the inner surface (and in some embodiments, the outer surface as well) of the prosthetic liner 100 receive and collect liquid droplets (e.g., sweat droplets) secreted by the user. Due to the low surface tension of the material of microchannels 102, forces of adhesion introduced by the microchannels 102 are greater than other forces, including those pertaining to cohesion and gravity. In this regard, the liquid droplets will effectively cling to the interior walls of the microchannels 102. Furthermore, the upper surface of each liquid droplet (i.e., liquid-air interface) spreads out in a concave-like shape in which the height of the liquid droplet at the point of contact with the interior walls of the microchannels 102 is higher than liquid fluids at the middle of the microchannels 102. While the cohesion force between liquid molecules within a liquid droplet will tend to attract to each other, it is recognized in the present disclosure that the liquid molecules will be directed along, drawn, and/or “climb” (e.g., upwards or against gravity) the microchannels 102 at a steady rate (e.g., where both adhesion and cohesion forces are equal). In preferred embodiments, the microchannels 102 will be formed in such a way that the liquid droplets will be directed upwards (e.g., in a direction from the bottom section 202 to the top section 206 of the prosthetic liner 100). It is to be understood, however, that the microchannels 102 may be formed in other ways and/or orientations so as to direct liquid droplets in other directions without departing from the teachings of the present disclosure.

In example embodiments in which the microchannels 102 direct liquid upwards, at least a portion of the top section 206 of the prosthetic liner 100 may include a liquid-retentive portion. The liquid-retentive portion may be formed using and/or having liquid absorbent and/or liquid retentive material, or the like, which may function to collect liquid (e.g., sweat, moisture, etc.) which has been collected by and travelled along the microchannels 102 throughout the day. For example, some, most, or all of the top section 206 of the prosthetic liner 100 may include liquid absorbent or retentive fabric, polymers, or the like (e.g., super absorbent polymers, or SAPs). In other embodiments, the liquid-retentive portion may be provided in other parts of the prosthetic liner 100, such as near the bottom section 202 of the prosthetic liner 100 and/or at one or more locations between the top section 206 and the bottom section 202 of the prosthetic liner 100.

In some embodiments, other suitable methods may be used to direct liquid towards the liquid-retentive portion (which may be provided in the top section 206, bottom section 202, and/or other parts of the prosthetic liner 100). Such suitable methods may include, but are not limited to, the use of pumps, negative pressure (e.g., vacuum, valves), other hydrophilic materials, formations, and/or assemblies, or the like.

As another example, for prosthetic limbs that regularly come into contact with other body parts (e.g., prosthetic limbs for arms or legs that come into contact with one or more other body parts when the user bends or moves a joint (e.g., an elbow, shoulder, or knee (each as applicable))), the portion(s) of the top section 206 of the prosthetic liner 100 that regularly come into contact with other body parts may not include liquid absorbing or retentive material since such contact with other body parts may cause collected liquids to be released. It is recognized in the present disclosure that such an embodiment prevents moisture buildup within and/or at the bottom section 202 of the prosthetic liner, which would otherwise cause discomfort and other negative effects to the user.

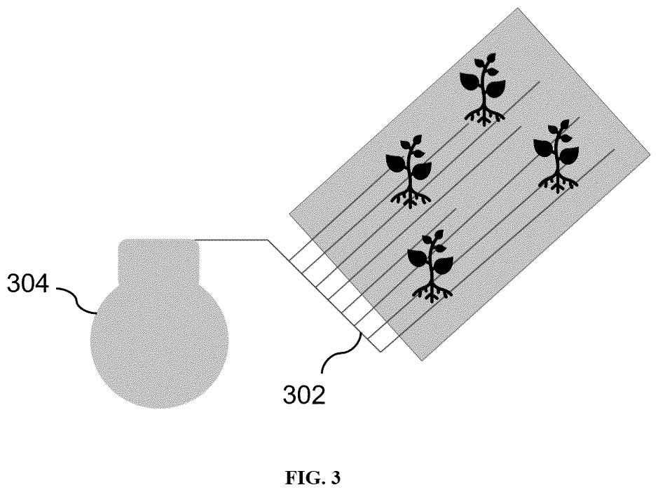

FIG. 3 illustrates another example embodiment for use in water systems such as drip irrigation.

Conventional irrigation drip systems use thin spaghetti type tubes to distribute water at a slow rate. These systems allow more control over water and nutrient supply, can be scaled for growth, are affordable to install, and are less likely to fail as compared to other irrigation methods. However, conventional irrigation drip systems may be complicated to install and require maintenance and/or replacement of pumps. Further, non-recovery drip systems result in wasted water and drip systems with recovery systems are high maintenance.

Example embodiments of the microchannel 302 are configurable or configured to direct, collect, and/or otherwise control water from the water source 304 to the plants. Similar to the microchannels 102 described above for prosthetic liners 100, the microchannels 302 may be configured to carry water upwards towards plants that may be above the water source 304. This method obviates the need for a pump and maintenance thereof and is easier to install. This system may be used for hydroponic set ups. Furthermore, this system may be a self-sufficient watering system if the water is recycled.

While various aspects and embodiments have been disclosed herein, it will be appreciated by a person skilled in the art that several of the above-disclosed structures, parameters, or processes thereof, can be desirably modified, adapted, and combined into alternative structures, processes and/or applications. The various aspects and embodiments disclosed herein are for purposes of illustration and are not intended to be limiting, with the true scope of the disclosure being indicated by the claims.

Various terms used herein have special meanings within the present technical field. Whether a particular term should be construed as such a “term of art” depends on the context in which that term is used. Terms are to be construed in light of the context in which they are used in the present disclosure and as one of ordinary skill in the art would understand those terms in the disclosed context. Definitions provided herein are not exclusive of other meanings that might be imparted to those terms based on the disclosed context.

Words of comparison, measurement, and timing such as “at the time”, “equivalent”, “during”, “complete”, and the like should be understood to mean “substantially at the time”, “substantially equivalent”, “substantially during”, “substantially complete”, etc., where “substantially” means that such comparisons, measurements, and timings are practicable to accomplish the implicitly or expressly stated desired result.

Additionally, the section headings and topic headings herein are provided for consistency with the suggestions under various patent regulations and practice, or otherwise to provide organizational cues. These headings shall not limit or characterize the embodiments set out in any claims that may issue from this disclosure. Specifically, a description of a technology in the “Background” is not to be construed as an admission that technology is prior art to any embodiments in this disclosure. Furthermore, any reference in this disclosure to “invention” in the singular should not be used to argue that there is only a single point of novelty in this disclosure. Multiple inventions may be set forth according to the limitations of the claims issuing from this disclosure, and such claims accordingly define the invention(s), and their equivalents, that are protected thereby. In all instances, the scope of such claims shall be considered on their own merits in light of this disclosure but should not be constrained by the headings herein.

What is claimed is:

1. A prosthetic liner for a prosthetic limb having an interior surface forming an interior cavity, the prosthetic liner comprising:
a liner body, the liner body formed in a shape resembling a shape of at least a portion of the interior surface of the prosthetic limb, the liner body having:
a first end for receiving an insertion of a body part of a user, wherein at least a portion of the first end of the liner body includes a liquid-retentive portion.

2. The prosthetic liner according to claim 1, wherein the liquid-retentive portion is formed using liquid absorbent or liquid retentive materials.

3. The prosthetic liner according to claim 2, wherein the liquid-retentive portion includes super absorbent polymers.

4. The prosthetic liner according to claim 1, further comprising a plurality of microchannels formed along an inner surface of the liner body, the plurality of microchannels connected at one end to the liquid-retentive portion of the first end of the liner body, wherein at least one of the plurality of microchannels is configured to receive liquid droplets and direct the received liquid droplets to the liquid-retentive portion of the first end of the liner body.

5. A prosthetic liner for a prosthetic limb, the prosthetic limb having an interior surface forming an interior cavity, the prosthetic liner comprising:
a liner body, the liner body formed in a shape resembling a shape of at least a portion of the interior surface of the prosthetic limb, the liner body configured to be housed in the interior cavity of the prosthetic limb, the liner body having:
a first end for receiving an insertion of a body part of a user, wherein at least a portion of the first end of the liner body includes a liquid-retentive portion;
a second end opposite to the first end;
an inner surface formed between the first and second ends, the inner surface of the liner body for contacting with at least a portion of the body part of the user, the inner surface of the liner body including:
a plurality of microchannels, the plurality of microchannels formed along the inner surface of the liner body, the plurality of microchannels connected at one end to the liquid-retentive portion of the first end of the liner body, wherein at least one of the plurality of microchannels is configured to receive liquid droplets and direct the received liquid droplets to the liquid-retentive portion of the first end of the liner body; and
an outer surface opposite to the inner surface of the liner body, the outer surface of the liner body facing the interior surface of the prosthetic limb and provided in the interior cavity of the prosthetic limb.

6. The prosthetic liner according to claim 5, wherein at least one of the following apply:
each of the plurality of microchannels has a width of not more than 0.2 mm; and
each of the plurality of microchannels is formed using soft hydrophilic material.

7. The prosthetic liner according to claim 5, wherein the liquid-retentive portion is formed using liquid absorbent or liquid retentive materials.

8. The prosthetic liner according to claim 7, wherein the liquid-retentive portion includes super absorbent polymers.

9. The prosthetic liner according to claim 5, wherein at least a portion of the second end of the liner body includes a second liquid-retentive portion.

10. The prosthetic liner according to claim 9, wherein the second liquid-retentive portion is formed using liquid absorbent or retentive materials.

11. The prosthetic liner according to claim 10, wherein the second liquid-retentive portion includes super absorbent polymers.

12. The prosthetic liner according to claim 9, wherein:
at least one of the plurality of microchannels is connected at another end to the second liquid-retentive portion; and
the at least one of the plurality of microchannels is configured to receive liquid droplets and direct the received liquid droplets to the second liquid-retentive portion.

13. The prosthetic liner according to claim 5, wherein the outer surface of the liner body includes:
a plurality of second microchannels, the plurality of second microchannels formed along the outer surface of the liner body, the plurality of second microchannels connected at one end to the liquid-retentive portion of the first end of the liner body, wherein at least one of the plurality of second microchannels is configured to receive liquid droplets and direct the received liquid droplets to the liquid-retentive portion of the first end of the liner body.

14. The prosthetic liner according to claim 13, wherein at least one of the following apply:
each of the plurality of second microchannels has a width of not more than 0.2 mm; and
each of the plurality of second microchannels is formed using soft hydrophilic material.

15. A prosthetic liner for a prosthetic limb, the prosthetic limb having an interior surface forming an interior cavity, the prosthetic liner comprising:
a liner body, the liner body formed in a shape resembling a shape of at least a portion of the interior surface of the prosthetic limb, the liner body configured to be housed in the interior cavity of the prosthetic limb, the liner body having:
a first end for receiving an insertion of a body part of a user;
a second end opposite to the first end;
an inner surface formed between the first and second ends, the inner surface of the liner body for contacting with at least a portion of the body part of the user, the inner surface of the liner body including:
a plurality of microchannels, the plurality of microchannels formed along the inner surface of the liner body, wherein at least one of the plurality of microchannels is configured to receive and collect liquid droplets; and
an outer surface opposite to the inner surface of the liner body, the outer surface of the liner body facing the interior surface of the prosthetic limb and provided in the interior cavity of the prosthetic limb, the outer surface of the liner body including:
a plurality of second microchannels, the plurality of second microchannels formed along the outer surface of the liner body, wherein at least one of the plurality of second microchannels is configured to receive and collect liquid droplets.

16. The prosthetic liner according to claim 15, wherein at least one of the following apply:
each of the plurality of microchannels has a width of not more than 0.2 mm;
each of the plurality of microchannels is formed using soft hydrophilic material;
each of the plurality of second microchannels has a width of not more than 0.2 mm; and
each of the plurality of second microchannels is formed using soft hydrophilic material.

17. The prosthetic liner according to claim 15, wherein at least a portion of the first end of the liner body includes a liquid-retentive portion, the liquid-retentive portion formed using liquid absorbent or liquid retentive materials.

18. The prosthetic liner according to claim 17, wherein:
at least one of the plurality of microchannels is connected at one end to the liquid-retentive portion; and
the at least one of the plurality of microchannels is configured to receive liquid droplets and direct the received liquid droplets to the liquid-retentive portion.

19. The prosthetic liner according to claim 17, wherein the liquid-retentive portion includes super absorbent polymers.

20. The prosthetic liner according to claim 15, wherein at least a portion of the second end of the liner body includes a second liquid-retentive portion.

21. The prosthetic liner according to claim 20, wherein the second liquid-retentive portion is formed using liquid absorbent or retentive materials.

22. The prosthetic liner according to claim 21, wherein the second liquid-retentive portion includes super absorbent polymers.

23. The prosthetic liner according to claim 20, wherein:
at least one of the plurality of microchannels is connected at another end to the second liquid-retentive portion; and
the at least one of the plurality of microchannels is configured to receive liquid droplets and direct the received liquid droplets to the second liquid-retentive portion.
